(19) United States
(12) Patent Application Publication
(10) Pub. No.: US 2026/0240601 A1
(43) Pub. Date: Aug. 20, 2026

(54) GUIDANCE OF OFF-AXIS SURGICAL INSTRUMENTS

(71) Applicants: Smith & Nephew, Inc., Memphis, TN (US); Smith & Nephew Orthopaedics AG, Zug (CH); Smith & Nephew Asia Pacific Pte. Limited, Singapore (SG)

(72) Inventors: Carolina DOS SANTOS RAPOSO, Coimbra (PT); João Pedro DE ALMEIDA BARRETO, Coimbra (PT); João Pedro SILVA NEVES, Figueira da Foz (PT); Diogo Emanuel RIBAS VAZ, Miranda do Corvo (PT)

(73) Assignees: Smith & Nephew, Inc., Memphis, TN (US); Smith & Nephew Orthopaedics AG, Zug (CH); Smith & Nephew Asia Pacific Pte. Limited, Singapore (SG)

(21) Appl. No.: 19/642,996

(22) Filed: Apr. 9, 2026

Related U.S. Application Data
(63) Continuation of application No. PCT/US2024/052750, filed on Oct. 24, 2024.
(60) Provisional application No. 63/602,953, filed on Nov. 27, 2023.

(51) Int. Cl. A61B 34/10; A61B 17/17; A61B 34/00

(52) U.S. Cl. CPC A61B 34/10; A61B 17/1739; A61B 34/25; A61B 2034/107

(57) ABSTRACT

A guidance system for performing alignment of an off-axis surgical instrument includes a surgical system configured to receive first data indicating a location of a planned tunnel through a surgical site, receive second data indicating a position of the surgical instrument relative to the planned tunnel at the surgical site, and, based on the first data and the second data, cause a display to present a visual representation indicative of a current position of a portion of the surgical instrument relative to the planned tunnel, a first rotational orientation of the aimer tool relative to the planned tunnel, and a second rotational orientation of the aimer tool relative to the planned tunnel.

### CROSS-REFERENCE TO RELATED APPLICATIONS

This application is a continuation and claims the benefit of International Application No. PCT/US2024/052750 filed October 24, 2024. PCT/US2024/052750 claims priority to U.S. Provisional Patent Application No. 63/602,953, entitled “Guidance of Off-Axis Surgical Instruments,” filed November 27, 2023. The entire disclosures of these applications are incorporated herein by reference.

### FIELD

The present disclosure relates to preoperative and intraoperative surgical analysis and processing, and, more particularly, to locating and forming tunnels for a surgical procedure.

### BACKGROUND

The background description provided here is for the purpose of generally presenting the context of the disclosure. Work of the presently named inventors, to the extent it is described in this background section, as well as aspects of the description that may not otherwise qualify as prior art at the time of filing, are neither expressly nor impliedly admitted as prior art against the present disclosure.

The Anterior Cruciate Ligament (ACL) is one of the key ligaments that provide stability to the knee joint. Playing sports that involve sudden stops or changes in direction is one of the main causes for ACL injury, an example of which is its complete tear. For this reason, an ACL tear is a common medical condition with more than 200,000 annual cases per year in the United States alone. A standard treatment may include arthroscopic reconstruction. During arthroscopic reconstruction, the torn ligament is replaced by a tissue graft that is pulled into the knee joint through tunnels opened with a drill in both the femur and tibia. Opening these tunnels in an anatomically correct position ensures knee stability and patient satisfaction, though the current failure rates in primary ACL reconstructions range from 10-15%.

### SUMMARY

A guidance system for performing alignment of an off-axis surgical instrument includes a surgical system configured to receive first data indicating a location of a planned tunnel through a surgical site, receive second data indicating a position of the surgical instrument relative to the planned tunnel at the surgical site, and, based on the first data and the second data, cause a display to present a visual representation indicative of a current position of a portion of the surgical instrument relative to the planned tunnel, a first rotational orientation of the aimer tool relative to the planned tunnel, and a second rotational orientation of the aimer tool relative to the planned tunnel.

In other aspects, one or more methods may include steps corresponding to the functions performed by the systems described herein. In other aspects, a processor may be configured to execute instructions to perform functions of the systems described herein.

Further areas of applicability of the present disclosure will become apparent from the detailed description, the claims and the drawings. The detailed description and specific examples are intended for purposes of illustration only and are not intended to limit the scope of the disclosure.

### BRIEF DESCRIPTION OF THE DRAWINGS

For a detailed description of example embodiments, reference will now be made to the accompanying drawings in which:

FIG. 1 is a block diagram of an example configuration within which the systems and methods disclosed herein could be implemented according to some embodiments of the present disclosure;

FIG. 2 is a block diagram illustrating components of an exemplary system according to some embodiments of the present disclosure;

FIGS. 3A-3E illustrate an example alignment process for an off-axis elbow aimer tool according to some embodiments of the present disclosure;

FIG. 4 illustrates steps of an example method for aligning an off-axis elbow aimer tool according to some embodiments of the present disclosure; and

FIG. 5 shows an example computer system or computing device configured to implement the various systems and methods of the present disclosure.

In the drawings, reference numbers may be reused to identify similar and/or identical elements.

### DEFINITIONS

Various terms are used to refer to particular system components. Different companies may refer to a component by different names – this document does not intend to distinguish between components that differ in name but not function. In the following discussion and in the claims, the terms “including” and “comprising” are used in an open-ended fashion, and thus should be interpreted to mean “including, but not limited to… .” Also, the term “couple” or “couples” is intended to mean either an indirect or direct connection. Thus, if a first device couples to a second device, that connection may be through a direct connection or through an indirect connection via other devices and connections.

Similarly, spatial and functional relationships between elements (for example, between device, modules, circuit elements, etc.) are described using various terms, including “connected,” “engaged,” “coupled,” “adjacent,” “next to,” “on top of,” “above,” “below,” and “disposed.” Unless explicitly described as being “direct,” when a relationship between first and second elements is described in the above disclosure, that relationship can be a direct relationship where no other intervening elements are present between the first and second elements, but can also be an indirect relationship where one or more intervening elements are present (either spatially or functionally) between the first and second elements. Nevertheless, this paragraph shall serve as antecedent basis in the claims for referencing any electrical connection as “directly coupled” for electrical connections shown in the drawing with no intervening element(s).

Terms of degree, such as “substantially” or “approximately,” are understood by those skilled in the art to refer to reasonable ranges around and including the given value and ranges outside the given value, for example, general tolerances associated with manufacturing, assembly, and use of the embodiments. The term “substantially,” when referring to a structure or characteristic, includes the characteristic that is mostly or entirely present in the characteristic or structure. As one example, numerical values that are described as “approximate” or “approximately” as used herein may refer to a value within +/- 5% of the stated value.

“A”, “an”, and “the” as used herein refers to both singular and plural referents unless the context clearly dictates otherwise. By way of example, “a processor” programmed to perform various functions refers to one processor programmed to perform each and every function, or more than one processor collectively programmed to perform each of the various functions. To be clear, an initial reference to “a (referent)”, and then a later reference for antecedent basis purposes to “the (referent)”, shall not obviate the fact the recited referent may be plural.

In general, terminology may be understood at least in part from usage in context. For example, terms, such as “and”, “or”, or “and/or,” as used herein may include a variety of meanings that may depend at least in part upon the context in which such terms are used. Typically, “or” if used to associate a list, such as A, B or C, is intended to mean A, B, and C, here used in the inclusive sense, as well as A, B or C, here used in the exclusive sense. In addition, the term “one or more” as used herein, depending at least in part upon context, may be used to describe any feature, structure, or characteristic in a singular sense or may be used to describe combinations of features, structures or characteristics in a plural sense. Similarly, terms, such as “a,” “an,” or “the,” again, may be understood to convey a singular usage or to convey a plural usage, depending at least in part upon context. In addition, the term “based on” may be understood as not necessarily intended to convey an exclusive set of factors and may, instead, allow for existence of additional factors not necessarily expressly described, again, depending at least in part on context. As used herein, the phrase at least one of A, B, and C should be construed to mean a logical (A OR B OR C), using a non-exclusive logical OR, and should not be construed to mean “at least one of A, at least one of B, and at least one of C.”

The terms “input” and “output” when used as nouns refer to connections (e.g., electrical, software) and/or signals, and shall not be read as verbs requiring action. For example, a timer circuit may define a clock output. The example timer circuit may create or drive a clock signal on the clock output. In systems implemented directly in hardware (e.g., on a semiconductor substrate), these “inputs” and “outputs” define electrical connections and/or signals transmitted or received by those connections. In systems implemented in software, these “inputs” and “outputs” define parameters read by or written by, respectively, the instructions implementing the function. In examples where used in the context of user input, “input” may refer to actions of a user, interactions with input devices or interfaces by the user, etc.

“Controller,” “module,” or “circuitry” shall mean, alone or in combination, individual circuit components, an application specific integrated circuit (ASIC), a microcontroller with controlling software, a reduced-instruction-set computer (RISC) with controlling software, a digital signal processor (DSP), a processor with controlling software, a programmable logic device (PLD), a field programmable gate array (FPGA), or a programmable system-on-a-chip (PSOC), configured to read inputs and drive outputs responsive to the inputs.

As used to describe various surgical instruments or devices, such as a probe, the term “proximal” refers to a point or direction nearest a handle of the probe (e.g., a direction opposite the probe tip). Conversely, the term “distal” refers to a point or direction nearest the probe tip (e.g., a direction opposite the handle).

For the purposes of this disclosure, a non-transitory computer readable medium (or computer-readable storage medium/media) stores computer data, which data can include computer program code (or computer-executable instructions) that is executable by a computer, in machine-readable form. By way of example, and not limitation, a computer readable medium may comprise computer readable storage media, for tangible or fixed storage of data, or communication media for transient interpretation of code-containing signals. Computer readable storage media, as used herein, refers to physical or tangible storage (as opposed to signals) and includes without limitation volatile and non-volatile, removable and non-removable media implemented in any method or technology for the tangible storage of information such as computer-readable instructions, data structures, program modules or other data. Computer readable storage media includes, but is not limited to, RAM, ROM, EPROM, EEPROM, flash memory or other solid state memory technology, optical storage, cloud storage, magnetic storage devices, or any other physical or material medium which can be used to tangibly store the desired information or data or instructions and which can be accessed by a computer or processor.

For the purposes of this disclosure, the term “server” should be understood to refer to a service point that provides processing, database, and communication facilities. By way of example, and not limitation, the term “server” can refer to a single, physical processor with associated communications and data storage and database facilities, or it can refer to a networked or clustered complex of processors and associated network and storage devices, as well as operating software and one or more database systems and application software that support the services provided by the server. Cloud servers are examples.

For the purposes of this disclosure, a “network” should be understood to refer to a network that may couple devices so that communications may be exchanged, such as between a server and a client device or other types of devices, including between wireless devices coupled via a wireless network, for example. A network may also include mass storage, such as network attached storage (NAS), a storage area network (SAN), a content delivery network (CDN) or other forms of computer or machine-readable media, for example. A network may include the Internet, one or more local area networks (LANs), one or more wide area networks (WANs), wire‐line type connections, wireless type connections, cellular or any combination thereof. Likewise, sub‐networks, which may employ differing architectures or may be compliant or compatible with differing protocols, may interoperate within a larger network.

For purposes of this disclosure, a “wireless network” should be understood to couple client devices with a network. A wireless network may employ stand‐alone ad‐hoc networks, mesh networks, Wireless LAN (WLAN) networks, cellular networks, or the like. A wireless network may further employ a plurality of network access technologies, including Wi-Fi, Long Term Evolution (LTE), WLAN, Wireless Router (WR) mesh, or 2nd, 3rd, 4^{th} or 5^{th} generation (2G, 3G, 4G or 5G) cellular technology, mobile edge computing (MEC), Bluetooth, 802.11b/g/n, or the like. Network access technologies may enable wide area coverage for devices, such as client devices with varying degrees of mobility, for example. In short, a wireless network may include virtually any type of wireless communication mechanism by which signals may be communicated between devices, such as a client device or a computing device, between or within a network, or the like.

A computing device may be capable of sending or receiving signals, such as via a wired or wireless network, or may be capable of processing or storing signals, such as in memory as physical memory states, and may, therefore, operate as a server. Thus, devices capable of operating as a server may include, as examples, dedicated rack‐mounted servers, desktop computers, laptop computers, set top boxes, integrated devices combining various features, such as two or more features of the foregoing devices, or the like.

For purposes of this disclosure, a client (or consumer or user) device, referred to as user equipment (UE)), may include a computing device capable of sending or receiving signals, such as via a wired or a wireless network. A client device may, for example, include a desktop computer or a portable device, such as a cellular telephone, a smart phone, a display pager, a radio frequency (RF) device, an infrared (IR) device a Near Field Communication (NFC) device, a Personal Digital Assistant (PDA), a handheld computer, a tablet computer, a phablet, a laptop computer, a set top box, a wearable computer, smart watch, an integrated or distributed device combining various features, such as features of the forgoing devices, or the like.

In some embodiments, as discussed below, the client device can also be, or can communicatively be coupled to, any type of known or to be known medical device (e.g., any type of Class I, II or III medical device), such as, but not limited to, a MRI machine, CT scanner, Electrocardiogram (ECG or EKG) device, photopletismograph (PPG), Doppler and transmit-time flow meter, laser Doppler, an endoscopic device neuromodulation device, a neurostimulation device, and the like, or some combination thereof.

### DETAILED DESCRIPTION

The present disclosure will now be described more fully hereinafter with reference to the accompanying drawings, which form a part hereof, and which show, by way of non-limiting illustration, certain example embodiments. Subject matter may, however, be embodied in a variety of different forms and, therefore, covered or claimed subject matter is intended to be construed as not being limited to any example embodiments set forth herein; example embodiments are provided merely to be illustrative. Likewise, a reasonably broad scope for claimed or covered subject matter is intended. Among other things, for example, subject matter may be embodied as methods, devices, components, or systems. Accordingly, embodiments may, for example, take the form of hardware, software, firmware or any combination thereof (other than software per se). The following detailed description is, therefore, not intended to be taken in a limiting sense.

Throughout the specification and claims, terms may have nuanced meanings suggested or implied in context beyond an explicitly stated meaning. Likewise, the phrase “in one embodiment” as used herein does not necessarily refer to the same embodiment and the phrase “in another embodiment” as used herein does not necessarily refer to a different embodiment. It is intended, for example, that claimed subject matter include combinations of example embodiments in whole or in part.

The present disclosure is described below with reference to block diagrams and operational illustrations of methods and devices. It is understood that each block of the block diagrams or operational illustrations, and combinations of blocks in the block diagrams or operational illustrations, can be implemented by means of analog or digital hardware and computer program instructions. These computer program instructions can be provided to a processor of a general purpose computer to alter its function as detailed herein, a special purpose computer, ASIC, or other programmable data processing apparatus, such that the instructions, which execute via the processor of the computer or other programmable data processing apparatus, implement the functions/acts specified in the block diagrams or operational block or blocks. In some alternate implementations, the functions/acts noted in the blocks can occur out of the order noted in the operational illustrations. For example, two blocks shown in succession can in fact be executed substantially concurrently or the blocks can sometimes be executed in the reverse order, depending upon the functionality/acts involved.

The position and orientation of femoral or tibial tunnels for surgical procedures such as ACL reconstruction significantly impact the success of the surgery, motivating the need for a pre-operative plan for properly locating the tunnels. In order to determine the anatomically correct position of the tunnel, some surgeons rely on specific anatomical landmarks. However, these landmarks may not be reliable and may even not exist in some patients. In computer-assisted surgical procedures (e.g., replacement of the ACL, reduction of femoro-acetabular impingement, etc.), surgical guidance may be provided in an image of a surgical site (e.g., within an image of the anatomy of a patient) to obtain more accurate tunnel positions and/or to guide the surgeon throughout the surgical procedure.

For the particular case of tibial tunnel drilling for ACL reconstruction, the goal of the guidance is to align a virtual line that corresponds to an axis of a planned tunnel with a line that goes through an axis of rotation of a surgical instrument (e.g., an aimer) through which the guidewire will pass. Opening these tunnels is typically done using a two-step process. In a first step, a tip of the aimer is fixed on a location that provides a desired position for the tunnel. In a second step, the aimer is rotated to adjust a direction/orientation of the tunnel.

Two example types of aimer tools used for ACL reconstruction are tip and elbow aimers. With tip aimers, the tip is placed directly in the location of a desired exit point of the tunnel. Accordingly, a guidewire (e.g., a guidewire of a guidewire/bullet assembly) of the aimer is aligned with the tip of the aimer. Conversely, with elbow aimers (“off-axis” instruments), the tip is not aligned with the bullet through which the guidewire passes. Therefore, the tip is placed in a location posterior to the desired exit point, which is difficult to precisely define/locate. Accordingly, when using elbow aimers, once the tip is fixed on the tibial plateau, rotating the aimer around the tip causes the location of the exit point of the tunnel to change. For this reason, opening tunnels with elbow aimers is a more difficult task than with tip aimers (for which rotation of the aimer does not change the location of the exit point).

Elbow aimer guidance systems and methods according to the principles of the present disclosure are configured to implement surgical navigation (e.g., computer-aided surgery, or CAS) techniques to provide guidance for using off-axis instruments such as elbow aimers. As described below in more detail, guidance images/instructions to be followed by the user/surgeon are presented on a display (e.g., overlaying a scope view of the surgical site) to facilitate use of an elbow aimer to locate tibial tunnels.

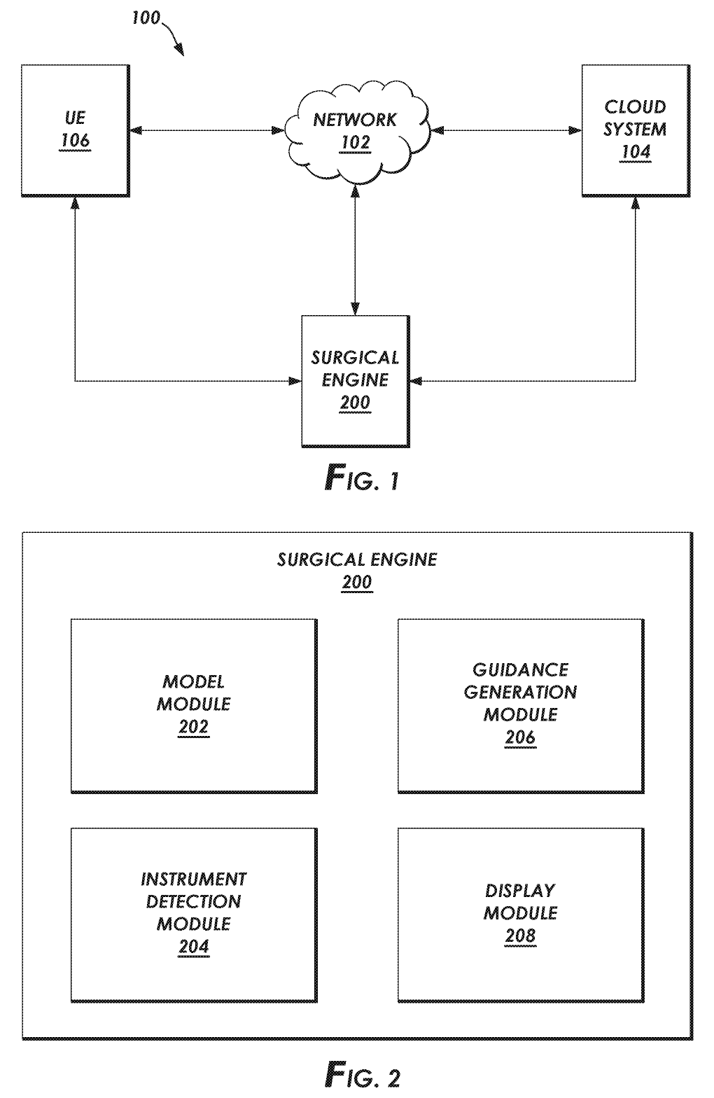

FIG. 1 shows an example system (or framework) 100 configured to implement one or more functions of the surgical navigation (e.g., elbow aimer guidance) systems and methods of the present disclosure. The system 100 includes a user device or user equipment (UE) 106, a network 102, a cloud system 104, and a surgical engine 200. The UE 106 can be any type of device, such as, but not limited to, a mobile phone, tablet, laptop, personal computer, sensor, Internet of Things (IoT) device, autonomous machine, and any other device equipped with a cellular, wireless, or wired transceiver. In some embodiments, as discussed above, the UE 106 can also be a medical device, or another device that is communicatively coupled to a medical device, that enables reception of readings from sensors of the medical device. For example, in some embodiments, the UE 106 can be a smartphone (or office/hospital equipment, for example) that is connected via WiFi, Bluetooth Low Energy (BLE) or NFC, for example, to a peripheral neuromodulation device. Thus, in some embodiments, the UE 106 can be configured to receive data from sensors associated with a medical device, as discussed in more detail below.

The network 102 can be any type of network, such as, but not limited to, a wireless network, cellular network, the Internet, a local-area network, or a wide-area network. As discussed herein, the network 102 can facilitate connectivity of the components of the system 100, as illustrated in FIG. 1.

The cloud system 104 can be any type of cloud operating platform and/or network based system upon which applications, operations, and/or other forms of network resources can be located. For example, system 104 can correspond to a service provider, network provider and/or medical provider from where services and/or applications can be accessed, sourced or executed from. In some embodiments, the cloud system 104 can include a server(s) and/or a database of information that is accessible over the network 102. In some embodiments, a database (not shown) of the system 104 can store a dataset of data and metadata associated with local and/or network information related to a user(s) of the UE 106, patients and the UE 106, and the services and applications provided by the cloud system 104 and/or the surgical engine 200.

The surgical engine 200, as discussed below in more detail, includes components configured to perform elbow aimer guidance techniques. Embodiments of how the engine 200 operates and functions, and the capabilities it includes and executes, among other functions, are discussed below in more detail.

According to some embodiments, the surgical engine 200 can be a special purpose machine or processor and could be hosted by a device on the network 102, within the cloud system 104 and/or on the UE 106. In some embodiments, the engine 200 can be hosted by a peripheral device connected to the UE 106 (e.g., a medical device, as discussed above).

According to some embodiments, the surgical engine 200 can function as an application provided by the cloud system 104. In some embodiments, the engine 200 can function as an application installed on the UE 106. In some embodiments, such application can be a web-based application accessed by the UE 106 over the network 102 from the cloud system 104 (e.g., as indicated by the connection between the network 102 and the engine 200, and/or the dashed line between the UE 106 and the engine 200 in FIG. 1). In some embodiments, the engine 200 can be configured and/or installed as an augmenting script, program or application (e.g., a plug-in or extension) to another application or program provided by the cloud system 104 and/or executing on the UE 106.

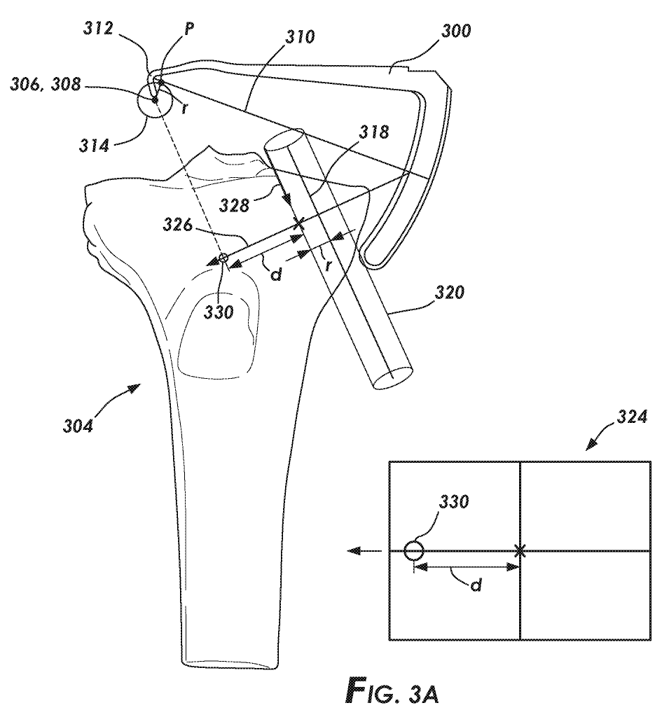

As illustrated in FIG. 2, according to some embodiments, the surgical engine 200 includes a model module 202, an instrument detection module 204, a guidance generation module 206, and a display module 208. It should be understood that the engine(s) and modules discussed herein are non-exhaustive, as additional or fewer engines and/or modules (or sub-modules) may be applicable to the embodiments of the systems and methods discussed. The operations, configurations, and functionalities of the engine 200 and each of the modules will be discussed below in more detail.

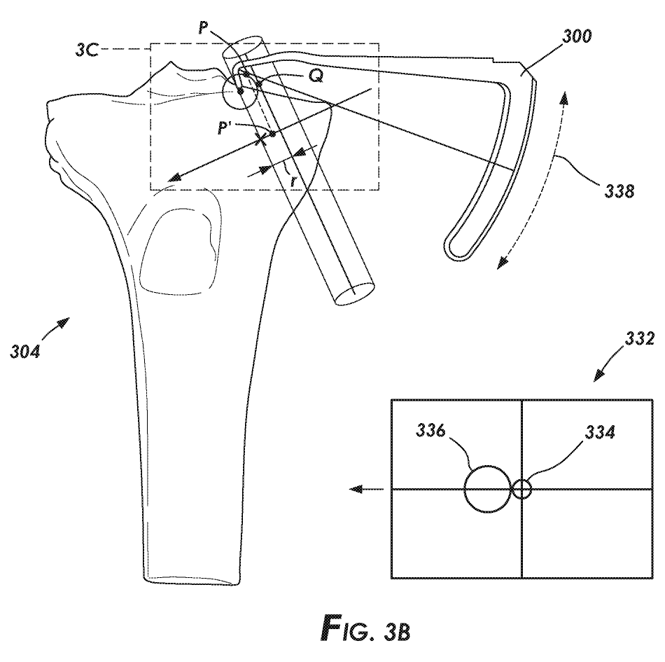

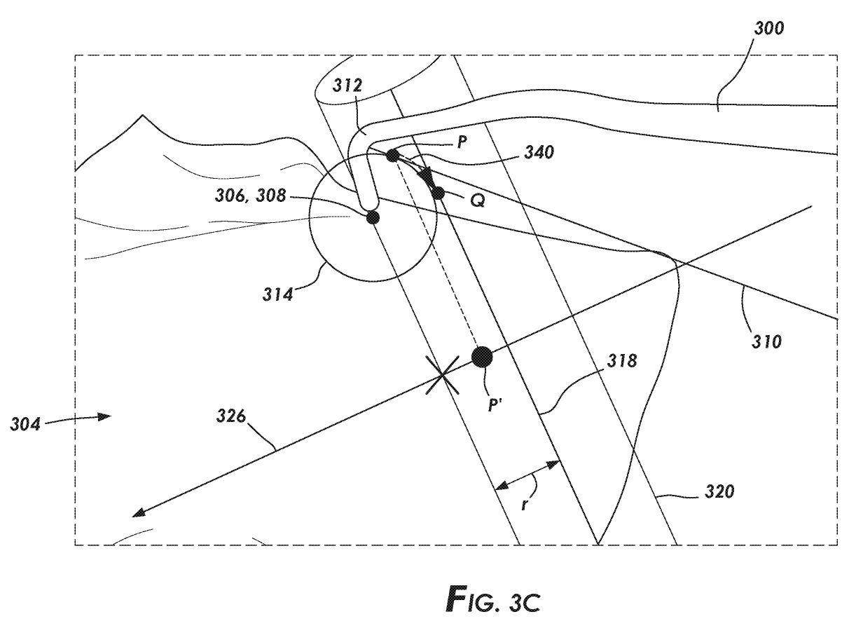

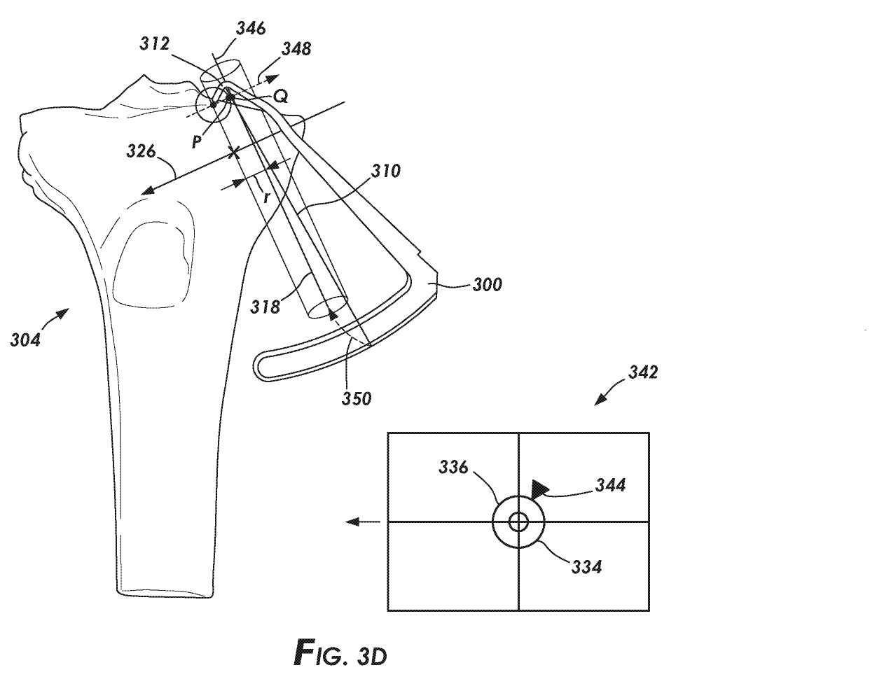

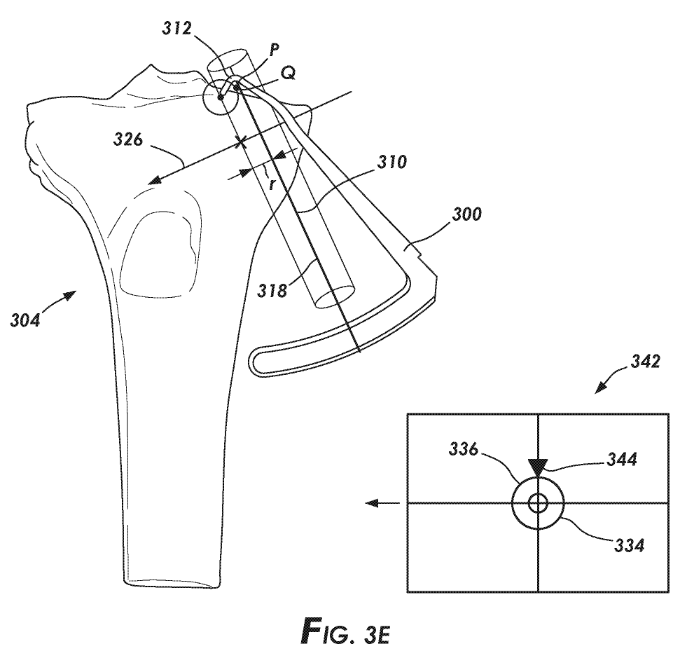

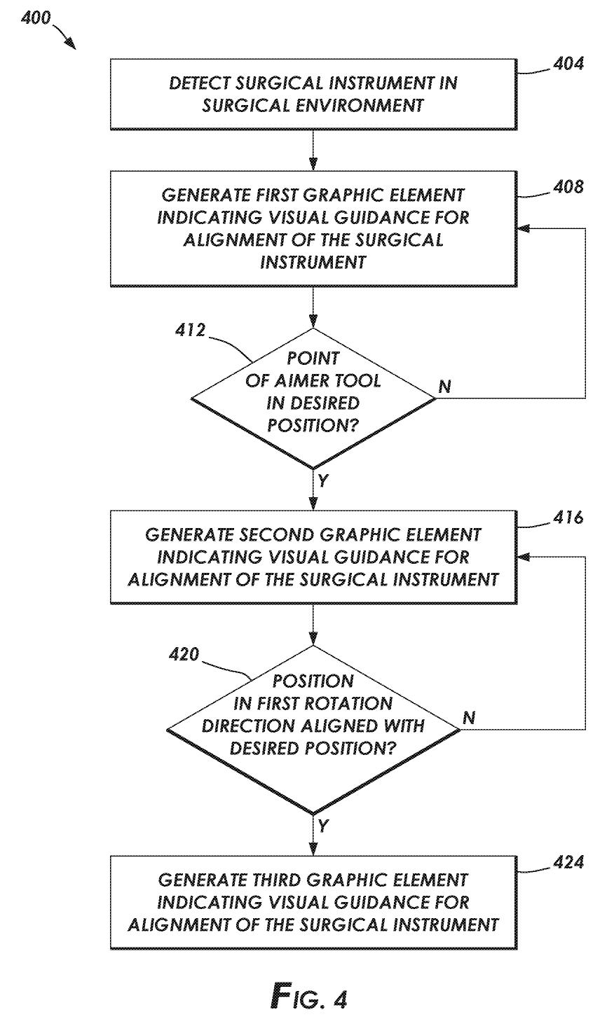

FIG. 3A illustrates example initial alignment of an off-axis elbow aimer tool 300 relative to a bone (e.g., represented by a bone model 304, such as a 3D bone model of a tibia) in accordance with the principles of the present disclosure. Although represented by the model 304 for illustration purposes, during alignment as described herein the aimer tool 300 may be positioned relative to actual patient anatomy (e.g., an end of a tibia or other bone for which tunnel placement is being prepared). In some examples, a model of the tibia, such as the model 304, may be also displayed (e.g., on a display of user equipment) for viewing by the surgeon to facilitate alignment. A model or graphical representation of the aimer tool 300 and the position/orientation of the aimer tool 300 relative to the model 304 may also be displayed for viewing by the surgeon. For example, the model module 202 of the surgical engine 200 may be configured to receive and/or generate the model 304, receive one or more inputs/requests to generate the model 304 or retrieve the model 304, process digital content corresponding to the model 304 for display, etc.

As shown, the aimer tool 300 includes a tip 306 identified by a point 308 and defines an axis 310. The axis 310 is aligned with an elbow 312 of the aimer tool 300 and may be referred to as a “central” axis of the aimer tool 300. For example, as shown, the axis 310 extends from an inner surface of the elbow 312 to an end of the aimer tool 300 opposite the elbow 312. With the tip 306 of the aimer tool 300 in a fixed location, rotating the aimer tool 300 about the point 308 in any direction changes an orientation of the axis 310 such that the axis 310 remains tangent to a sphere 314 centered on the point 308 (i.e., the tip of the aimer tool 300) and having a radius r. In other words, the radius r of the sphere 314 corresponds to a distance (e.g., a Euclidean distance) between the tip 306 of the aimer tool 300 and a point of tangency P on the axis 310. As another example, the radius r may be an orthogonal distance between the axis 310 and the center of the sphere 314.

FIG. 3A includes a planned tunnel 318, which, in some examples, may be displayed/overlaid on the model 304. The planned tunnel 318 may correspond to a planned location of the tunnel as determined pre-operatively. Accordingly, as used for display to the surgeon, implementation of the systems and methods described below, etc., the planned tunnel 318 corresponds to data (e.g., stored data) indicating a location of the planned tunnel 318 as previously obtained by the surgeon and/or other personnel. As used in this context, “previously” may correspond to immediately prior to performing alignment of the aimer 300 as described below. As described herein, the “location” of the planned tunnel 318 may correspond to a line or axis aligned with a center of the planned tunnel 318.

To align the axis 310 with a location/axis of the planned tunnel 318, the tip 306 is first moved to a location of an arbitrary point X on a surface of a cylinder 320 having the radius r and an axis of rotation corresponding to the planned tunnel 318. The point X may correspond to various points on the surface of the cylinder 320 corresponding to a distance r from the planned tunnel 318. This translational alignment of the tip 306 of the aimer tool 300 can be achieved by translating/moving the aimer tool 300 until the tip 306 is aligned with a point on the surface of the cylinder 320. Systems and methods according to the present disclosure are configured to provide, to the surgeon, visual guidance/instructions for moving the aimer tool 300 in the manner described above and in subsequent steps to complete alignment of the aimer tool 300 as described below in more detail.

As one example, the instrument detection module 204 is configured to detect the aimer tool 300 and a position/orientation of the aimer tool 300 in 3D space within the surgical environment. For example, the surgical environment may include one or more fiducials or other markers arranged in a fixed location (e.g., a bone fiducial anchored to patient anatomy). The aimer tool 300 can be tracked, using a camera or other imaging/sensing device, in accordance with relative 3D positions of the aimer tool 300 and the bone fiducial. The instrument detection module 204 is configured to determine the orientation of the aimer tool 300 relative to the patient anatomy based on the position tracked in this manner. Example systems and methods for tracking a surgical instrument relative to a bone fiducial are described in more detail in International Pat. App. No. PCT/US2024/046069, filed on 11 September, 2024, the entire contents of which are incorporated herein by reference. As generally described herein, detecting a position of the aimer tool 300 or a specific portion of the aimer tool 300, such as the point 306, includes generating, receiving, and/or otherwise obtaining data indicating the position of the aimer tool 300 within the surgical site, relative to patient anatomy, relative to the planned tunnel 318, etc.

The guidance generation module 206 according to the principles of the present disclosure is configured to generate, in one or more steps or stages, visual guidance/instructions for the surgeon. The visual guidance is calculated/generated based on the position (e.g., a current position) of the aimer tool 300 determined by the instrument detection module 204. The visual guidance may be adjusted, in real-time, as the aimer tool 300 is moved within the surgical environment.

The display module 208 is configured to cause the visual guidance (e.g., a visual guidance graphic element 324) to be displayed (e.g., on a display of the UE 106 and/or another computing device) for viewing by the surgeon. The graphic element 324 corresponds to a visual representation of relative current and desired or target positions of the aimer tool 300 as described below in more detail. In some examples, the graphic element 324 is displayed along with a visual representation of the aimer tool 300 and/or the model 304, the planned tunnel 318, etc. (e.g., as an overlay). In other examples, only the graphic element 324 is displayed. As shown, the graphic element 324 corresponds to a first guidance step.

As shown, the graphic element 324 corresponds to a reference frame or plane 326. The reference plane 326 is perpendicular to the planned tunnel 318, which extends in a direction normal to the reference plane 326. The graphic element 324 corresponds to the reference plane 326 as viewed from a direction indicated by arrow 328. An origin X of the reference plane 326 (e.g., an origin of a scope view) corresponds to the point X on the surface of the cylinder 320 (e.g. a target position X), which is offset from the axis of the planned tunnel 318 by the radius r as described above. Vertical and horizontal axes of the reference plane 326 as represented in the graphic element 324 correspond to coronal and sagittal directions, respectively. A current position of the tip 306 of the aimer tool 300 is shown at 330. Accordingly, in a first guidance step, the graphic element 324 displays, to the surgeon, the current position 330 of the tip 306, a target position X of the tip 306, and an offset distance d between the current position 330 and the target position X. Alignment of the tip 306 is achieved by translating the aimer tool 300 until the representation of the position of the tip 330 is aligned with the origin/target position X. The guidance may include the graphic element 324, additional visual instructions (e.g., arrows indicating a desired movement direction of the aimer tool 300), numerical values indicative of the distance d, audio instructions, or combinations thereof. Although shown as a circle and an “X” in FIG. 3A, other graphical elements may be used to represent the current position 330 of the tip 306, the target position X, etc.

FIGS. 3B and 3C illustrate alignment of the aimer tool 300 subsequent to the translational alignment achieved as described above in FIG. 3A. For example, the surgical engine 200 may be configured to detect when the aimer tool 300 is aligned such that the tip 306 is aligned with the target position X and advance an application/system state to a state corresponding to a second guidance step or stage.

With the tip 306 aligned as described above, various rotations of the aimer tool 300 may be performed (e.g., by the surgeon) such that the axis 310 of the aimer tool 300 is aligned with the planned tunnel 318. In other words, one or more rotational orientations of the aimer tool 300 may be adjusted. For example, as shown in FIG. 3B (and, in FIG. 3C, an inset of a portion of FIG. 3B), the planned tunnel 318 is tangent to a point Q on the sphere 314.

In a graphic element 332 corresponding to the second guidance step, an origin of the reference plane or scope view, as shown at 334, corresponds to the point Q (or, a projection of the point Q onto the reference frame 326. A point P’ corresponds to a projection of the point of tangency P onto the reference frame 326. The point P’ is represented graphically at 336 in the graphic element 332. Rotation of the aimer tool 300 in a first rotation direction (e.g., a first angular deviation) shown by arrow 338 and about an axis defined by the point 308 causes the point of tangency P to move in a direction shown by arrow 340. As this rotation of the aimer tool 300 is tracked, the point P’ (and the graphical representation 336 of the point P’) moves relative to the origin 334 and the point Q. Accordingly, the graphic element 336 provides visual guidance to the surgeon as the aimer tool 300 is rotated until the graphic element 336 is aligned with (e.g., centered with) the origin 334. Although shown circles in FIG. 3B, other graphical elements may be used to represent the position of the point P’, the origin or target position 334, etc. A rotational orientation of the aimer tool 300 in the first rotation direction may be referred to as a first rotational orientation.

FIG. 3D illustrates, in a graphic element 342 corresponding to a third guidance step, the graphical element 336 aligned with the origin 334 subsequent to rotation of the aimer tool 300 by the surgeon. In other words, the points P and Q are also aligned. However, the aimer tool 300 (e.g., the axis 310 may nonetheless not be aligned with the planned tunnel 318 in another (e.g., second) rotation direction (e.g., a second angular deviation). Accordingly, the graphic element 342 may include an arrowhead 344 or other indicator or graphical element indicating misalignment of the aimer tool 300 (e.g., the axis 310) relative to the planned tunnel 318 in the second rotation direction. For example, the second angular deviation corresponds to an angle between the axis 310 and the planned tunnel 318 in a plane (e.g., a plane 346) whose normal is a vector 348 that passes through the tip 306 and the point Q.

Accordingly, a third guidance step or stage includes guiding the surgeon to rotate the aimer tool 300 in a direction shown at 350 (i.e., rotation about/around an axis defined by the vector 348) until the arrowhead 344 is aligned with an alignment feature such as a vertical axis of the graphic element 342. For example, as shown in FIG. 3D, rotating the aimer tool 300 along the direction 350 causes the arrowhead 344 to rotate around the circular graphical element 336 toward (or away from) the vertical axis. In this guidance step, rotation of the aimer tool 300 in the direction 338 will cause the aimer tool 300 to become misaligned with the planned tunnel 318 in the first rotation direction (i.e., cause the graphical element 336 to move away from/become misaligned with the origin 334). Accordingly, during the third guidance step, the surgeon is provided visual guidance for aligning the aimer tool 300 in the second rotation direction while also maintaining the alignment of the aimer tool 300 in the first rotation direction. FIG. 3E shows the graphical element 342 with the desired alignment of the aimer tool in both the first and second rotation directions such that the graphical elements 334 and 336 are aligned (e.g., concentric) and the arrowhead 344 is aligned with a feature of graphic element 342, such as the vertical axis. Although shown as corresponding to the vertical axis, in other examples alignment may be indicated by other features, such as the horizontal axis or another feature. A rotational orientation of the aimer tool 300 in the second rotation direction may be referred to as a second rotational orientation.

As described herein, the arbitrary point X on the surface of the cylinder 320 is used. However, in various examples, a selected location of the point X may be constrained by specific patient anatomy. Accordingly, a set of valid 3D points may be restricted to points of intersection between the cylinder 320 and a surface of the model 304. For the particular case of the tibia, this intersection typically corresponds to a closed curve on the tibial plateau. Further, since this approach may be applied to minimally invasive surgeries, the aimer tool 300 will enter the joint through a portal, which is a small incision in the skin of the patient, and thus only a subset of the set of points may be accessible. By knowing the location of the portal with respect to the bone, the subset of reachable points can be estimated and used to determine the point that will be considered as the origin of the scope view. Determining such point may involve, for instance, considering a midway point of the subset, manually selecting a point from the subset, or using curvature information to select the point (e.g., choosing the point that is located in the region with a smallest curvature). Determining the subset of accessible points may involve (i) determining a plane that contains the portal and the planned tunnel, (ii) intersecting this plane with the set of all valid 3D points, (iii) choosing the most posterior point in case there is more than one intersection and (iv) considering only the points with an angular deviation (with respect to the point selected in (iii)) lower than a pre-defined threshold.

In case the location of the portal is not known, alternative approaches for selecting the origin of the scope view can be considered. For guiding an arbitrary tunnel, and considering that portals are opened in the most anterior part of the tibia, the origin may be set as the point of the set of valid 3D points that is most posterior. Further, when the tunnel is determined using an aimer, the origin of the scope view may be defined by (i) determining a plane that best fits all the aimer axes and the aimer tip, (ii) intersecting this plane with the set of all valid 3D points, and (iii) choosing the most posterior point in case there is more than one intersection.

The systems and methods of the present disclosure are described for the particular case of an elbow aimer but may also be for any surgical tool that has an axis to be guided and a tip that is not aligned with the axis (e.g., “off-axis” surgical instruments or tools).

As another example, when the tip 306 is not aligned, the planned tunnel 318 may not be tangent to the sphere 314. In this case, the planned tunnel 318 either intersects the sphere 314 at two points or does not intersect the sphere 314. In situations where the planned tunnel 318 intersects the sphere 314 at two points, the point Q can be selected as the point in the sphere 314 that is closest to a centroid of the two intersection points. In situations where the planned tunnel 318 does not intersect the sphere 314, the point Q may be selected as the point in the sphere 314 that is nearest to the planned tunnel 318. These algorithmic options allow for an intuitive behavior of circular indicators in the scope view, improving the usability of the system.

In some examples, the proposed systems and methods may be used with CAS systems that implement any sensing modality such as visual, optical, and/or electromagnetic tracking.

Although described above as a process including three guidance steps, the principles of the present disclosure may be implemented as a process that includes fewer than or more than three guidance steps (e.g., a single guidance step having a guidance graphic element that includes/presents visual indicators for tip position as well both rotation directions (e.g., multiple circular indicators, the arrowhead, etc.).

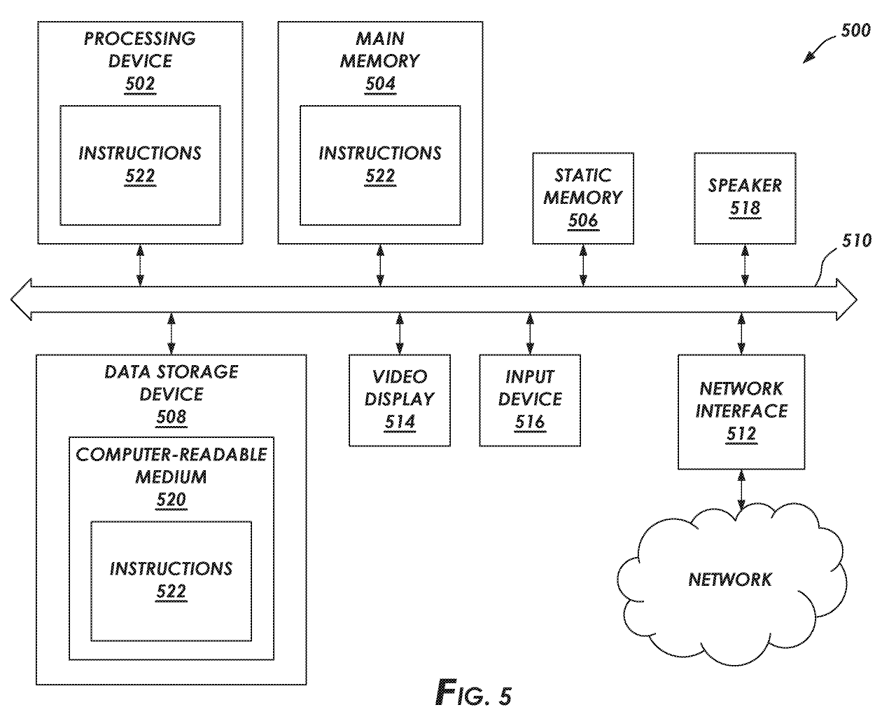

FIG. 4 illustrates an example method 400 for aligning an off-axis elbow aimer tool, such as the aimer tool 300, according to the principles of the present disclosure. The method 400 may be performed by one or more computing devices, processors or processing devices, the surgical engine 200, a surgical system (e.g., a surgical navigation system), a computer system 500 described below in more detail, etc. At least a portion of the method 400 may be performed using a user device or equipment, such as a tablet or other computing device including a user interface, display, etc. Accordingly, portions of the method 400 correspond to providing visual and/or audio guidance may be performed/implemented by a device including a user interface. Further, the method 400 as described below assumes additional steps/functions that may be performed prior to, during, and/or subsequent to the method 400 (e.g., other pre-, intra-, and/or post-operative steps).

At 404, the method 400 includes detecting a surgical instrument in a surgical environment, such as detecting a position/orientation of an elbow aimer tool relative to patient anatomy (e.g., a tibia or other anatomical structure). Detecting the surgical instrument may include using a camera to detect the position of the aimer tool within a view of a scope or other imaging device.

At 408, the method 400 includes generating and providing (e.g., displaying), on a display of a computing device, a first graphic element indicating first visual guidance for movement/alignment of the aimer tool relative to the patient anatomy. The first graphic element includes, based on a detected current position of the aimer tool, visual indicators that indicate a current position and a desired position of a portion of the aimer tool, such as a point of the aimer tool. In one example, the desired position is determined in accordance with at least one of a location of a planned tunnel, an axis of the planned tunnel, and a sphere having a radius based on the tip of the aimer tool and an axis defined by the aimer tool. Generating the first graphic element may correspond to techniques described above with respect to FIG. 3A.

At 412, the method 400 includes determining whether the point of the aimer tool is in the desired position (e.g., by using instrument detection techniques as described herein, in response to user input, determining whether respective visual indicators of the current position and the desired position are aligned in the first graphic element, etc.). If true, the method 400 continues to 416. If false, the method 400 continues to display the first graphic element.

At 416, the method 400 includes generating and providing a second graphic element indicating second visual guidance for movement of the aimer tool relative to the patient anatomy. The second graphic element includes, based on a detected current position of the aimer tool, visual indicators that indicate a current position of the aimer tool in a first rotation direction and a desired position of the aimer tool in the first rotation direction. In one example, the desired position in the first rotation direction is determined in accordance with at least one of a location of a planned tunnel, an axis of the planned tunnel, a sphere having a radius based on the tip of the aimer tool and the axis defined by the aimer tool, a point of tangency to the sphere on the axis of the aimer tool, and a point of tangency to the sphere on the location of the planned tunnel (e.g., on the axis defined by the planned tunnel). Generating the second graphic element may correspond to techniques described above with respect to FIGS. 3B and 3C.

At 420, the method 400 includes determining whether the position of the aimer tool in the first rotation direction is aligned with the desired position in the first rotation direction. If true, the method 400 continues to 424. If false, the method 400 continues to display the second graphic element.

At 424, the method 400 includes generating and providing a third graphic element indicating second visual guidance for movement of the aimer tool relative to the patient anatomy. The third graphic element includes, based on a detected current position of the aimer tool, visual indicators that indicate a current position of the aimer tool in a second rotation direction and a desired position of the aimer tool in the second rotation direction. In one example, the desired position in the second rotation direction is determined in accordance with at least one of the location of a planned tunnel, the axis of the planned tunnel, and the axis defined by the aimer tool. A relationship between the visual indicators for the current position and the desired position may be determined in accordance with an angle between the axis of the planned tunnel and the axis defined by the aimer tool. Generating the third graphic element may correspond to techniques described above with respect to FIGS. 3D and 3E.

FIG. 5 shows an example computer system or computing device 500 configured to implement the various systems and methods of the present disclosure. In one example, the computer system 500 may correspond to one or more computing devices of the system 100, the surgical engine 200, a tablet device within a surgical room, or any other system that implements any or all the various methods discussed in this specification. For example, the computer system 500 may be configured to implement all or portions of the method 400. The computer system 500 may be connected (e.g., networked) to other computer systems in a local-area network (LAN), an intranet, and/or an extranet, or at certain times the Internet (e.g., when not in use in a surgical procedure). The computer system 500 may be a server, a personal computer (PC), a tablet computer or any device capable of executing a set of instructions (sequential or otherwise) that specify actions to be taken by that device. Further, while only a single computer system is illustrated, the term “computer” shall also be taken to include any collection of computers that individually or jointly execute a set (or multiple sets) of instructions to perform any one or more of the methods discussed herein.

The computer system 500 includes a processing device 502, a main memory 504 (e.g., read-only memory (ROM), flash memory, dynamic random access memory (DRAM) such as synchronous DRAM (SDRAM)), a static memory 506 (e.g., flash memory, static random access memory (SRAM)), and a data storage device 508, which communicate with each other via a bus 510.

The processing device 502 represents one or more general-purpose processing devices such as a microprocessor, central processing unit, or the like. More particularly, the processing device 502 may be a complex instruction set computing (CISC) microprocessor, reduced instruction set computing (RISC) microprocessor, very long instruction word (VLIW) microprocessor, or a processor implementing other instruction sets or processors implementing a combination of instruction sets. The processing device 502 may also be one or more special-purpose processing devices such as an application specific integrated circuit (ASIC), a field programmable gate array (FPGA), a digital signal processor (DSP), network processor, or the like. The processing device 502 is configured to execute instructions for performing any of the operations and steps discussed herein. Once programmed with specific instructions, the processing device 502, and thus the entire computer system 500, becomes a special-purpose device, such as the surgical engine 200.

The computer system 500 may further include a network interface device 512 for communicating with any suitable network. The computer system 500 also may include a video display 514, one or more input devices 516 (e.g., a microphone, a keyboard, and/or a mouse), and one or more speakers 518. In one illustrative example, the video display 514 and the input device(s) 516 may be combined into a single component or device (e.g., an LCD touch screen).

The data storage device 508 may include a computer-readable storage medium 520 on which the instructions 522 (e.g., implementing any methods and any functions performed by any device and/or component depicted described herein) embodying any one or more of the methodologies or functions described herein is stored. The instructions 522 may also reside, completely or at least partially, within the main memory 504 and/or within the processing device 502 during execution thereof by the computer system 500. As such, the main memory 504 and the processing device 502 also constitute computer-readable media. In certain cases, the instructions 522 may further be transmitted or received over a network via the network interface device 512.

While the computer-readable storage medium 520 is shown in the illustrative examples to be a single medium, the term “computer-readable storage medium” should be taken to include a single medium or multiple media (e.g., a centralized or distributed database, and/or associated caches and servers) that store the one or more sets of instructions. The term “computer-readable storage medium” shall also be taken to include any medium that is capable of storing, encoding or carrying a set of instructions for execution by the machine and that cause the machine to perform any one or more of the methodologies of the present disclosure. The term “computer-readable storage medium” shall accordingly be taken to include, but not be limited to, solid-state memories, optical media, and magnetic media.

The foregoing description is merely illustrative in nature and is in no way intended to limit the disclosure, its application, or uses. The broad teachings of the disclosure can be implemented in a variety of forms. Therefore, while this disclosure includes particular examples, the true scope of the disclosure should not be so limited since other modifications will become apparent upon a study of the drawings, the specification, and the following claims. It should be understood that one or more steps within a method may be executed in different order (or concurrently) without altering the principles of the present disclosure. Further, although each of the embodiments is described above as having certain features, any one or more of those features described with respect to any embodiment of the disclosure can be implemented in and/or combined with features of any of the other embodiments, even if that combination is not explicitly described. In other words, the described embodiments are not mutually exclusive, and permutations of one or more embodiments with one another remain within the scope of this disclosure.

What is claimed is:

1. A guidance system for performing alignment of an off-axis surgical instrument, the guidance system comprising:
a surgical system configured to
receive first data indicating a location of a planned tunnel through a surgical site,
receive second data indicating a position of the surgical instrument relative to the planned tunnel at the surgical site, and
based on the first data and the second data, cause a display to present a visual representation indicative of (i) a current position of a portion of the surgical instrument relative to the planned tunnel, (ii) a first rotational orientation of the aimer tool relative to the planned tunnel, and (iii) a second rotational orientation of the aimer tool relative to the planned tunnel.

2. The guidance system of claim 1, wherein the surgical instrument is an elbow aimer tool.

3. The guidance system of claim 2, wherein respective trajectories of a tip of the elbow aimer tool and a guidewire of the elbow aimer tool do not intersect.

4. The guidance system of claim 2, wherein the visual representation includes a first graphic element indicating a first target position of the portion of the elbow aimer tool and a current position of the portion of the elbow aimer tool.

5. The guidance system of claim 4, wherein the portion of the elbow aimer tool is a tip of the elbow aimer tool.

6. The guidance system of claim 5, wherein the first target position corresponds to a location offset from the location of the planned tunnel by a predetermined distance.

7. The guidance system of claim 6, wherein the predetermined distance corresponds to a radius of a sphere centered on the tip of the elbow aimer tool, wherein the radius corresponds to a distance between the tip of the elbow aimer tool and the guidewire.

8. The guidance system of claim 7, wherein the visual representation includes a second graphic indicating a second target position of the elbow aimer tool in a first rotational direction corresponding to the first rotational orientation and a current position of the elbow aimer tool in the first rotational direction.

9. The guidance system of claim 8, wherein the second target position corresponds to a point of tangency of the sphere on an axis of the planned tunnel.

10. The guidance system of claim 8, wherein the visual representation includes a third graphic indicating a third target position of the elbow aimer tool in a second rotational direction corresponding to the second rotational orientation and a current position of the elbow aimer tool in the second rotational direction.

11. The guidance system of claim 10, wherein the third target position corresponds to an axis of the planned tunnel.

12. A method for performing alignment of an off-axis surgical instrument, the method comprising:
receiving first data indicating a location of a planned tunnel through a surgical site;
receiving second data indicating a position of the surgical instrument relative to the planned tunnel at the surgical site; and
based on the first data and the second data, causing a display to present a visual representation indicative of (i) a current position of a portion of the surgical instrument relative to the planned tunnel, (ii) a first rotational orientation of the aimer tool relative to the planned tunnel, and (iii) a second rotational orientation of the aimer tool relative to the planned tunnel.

13. The method of claim 12, wherein the surgical instrument is an elbow aimer tool, and wherein the portion of the elbow aimer tool is a tip of the elbow aimer tool.

14. The method of claim 13, wherein the visual representation includes a first graphic element indicating a first target position of the tip of the elbow aimer tool and a current position of the tip of the elbow aimer tool.

15. The method of claim 14, wherein the first target position corresponds to a location offset from the location of the planned tunnel by a predetermined distance.

16. The method of claim 15, wherein the predetermined distance corresponds to a radius of a sphere centered on the tip of the elbow aimer tool, wherein the radius corresponds to a distance between the tip of the elbow aimer tool and the guidewire.

17. The method of claim 16, wherein the visual representation includes a second graphic indicating a second target position of the elbow aimer tool in a first rotational direction corresponding to the first rotational orientation and a current position of the elbow aimer tool in the first rotational direction.

18. The method of claim 17, wherein the second target position corresponds to a point of tangency of the sphere on an axis of the planned tunnel.

19. The method of claim 17, wherein the visual representation includes a third graphic indicating a third target position of the elbow aimer tool in a second rotational direction corresponding to the second rotational orientation and a current position of the elbow aimer tool in the second rotational direction.

20. The method of claim 19, wherein the third target position corresponds to an axis of the planned tunnel.
